(19) United States
(12) Patent Application Publication
(10) Pub. No.: US 2026/0026781 A1
(43) Pub. Date: Jan. 29, 2026

(54) ULTRASONIC CATHETER

(71) Applicant: TERUMO KABUSHIKI KAISHA, Tokyo (JP)

(72) Inventors: Hiroyuki ISHIHARA, Ashigarakami-gun (JP); Itaru OKUBO, Ashigarakami-gun (JP); Katsuhiko SHIMIZU, Fujinomiya-shi (JP); Shogo YAMAYA, Ueda-shi (JP)

(73) Assignee: TERUMO KABUSHIKI KAISHA, Tokyo (JP)

(21) Appl. No.: 19/343,061

(22) Filed: Sep. 29, 2025

Related U.S. Application Data
(63) Continuation of application No. PCT/JP2024/008071, filed on Mar. 4, 2024.

(30) Foreign Application Priority Data
Mar. 31, 2023 (JP) 2023-057057

(51) Int. Cl. A61B 8/12; A61B 8/00; A61M 25/00; A61M 25/01

(52) U.S. Cl. CPC A61B 8/12; A61B 8/445; A61M 25/0074; A61M 25/0113

(57) ABSTRACT

An ultrasound catheter includes: an elongated sheath; an ultrasonic vibrator disposed inside the sheath; and a drive shaft holding the ultrasonic vibrator inside the sheath and rotating the ultrasonic vibrator about a central axis of the sheath, the ultrasonic vibrator includes: an ultrasonic oscillation surface substantially parallel to the central axis of the sheath or inclined at an angle of 15 degrees or smaller with respect to the central axis of the sheath; a back surface that faces in a direction opposite to a direction in which the ultrasonic oscillation surface faces; and a side surface substantially orthogonal to the direction in which the ultrasonic oscillation surface faces, and, in the ultrasonic vibrator, an ultrasonic attenuation member covers the back surface and at least parts of the lateral-direction side surface portions that the side surface portion includes.

### CROSS-REFERENCES TO RELATED APPLICATIONS

This application is a continuation of International Patent Application No. PCT/JP2024/008071 filed on Mar. 4, 2024, which claims priority to Japanese Patent Application No. 2023-057057 filed on Mar. 31, 2023, the entire content of both of which is incorporated herein by reference.

### TECHNOLOGICAL FIELD

The present invention generally relates to an ultrasound catheter that is inserted into a lumen of a heart or a blood vessel, for example, to acquire an image.

### BACKGROUND DISCUSSION

When an affected part is examined from a heart or a blood vessel, for example, an ultrasound catheter that is inserted into a lumen of a living body, that utilizes ultrasonic waves, and that acquires an image is used. The ultrasound catheter includes a vibrator for transmitting and receiving ultrasonic waves, a drive shaft for rotating the vibrator, and a sheath for accommodating and allowing the vibrator and the drive shaft to rotate. The vibrator is rotated and driven by the drive shaft in the sheath and transmits and receives ultrasonic waves for acquiring an image inside of the living body.

The vibrator is disposed in a closed space in a distal end portion of the sheath and faces in a direction orthogonal to a central axis of the sheath or a direction slightly inclined from the direction. An example of such an ultrasound catheter is disclosed in WO 2020/196337 A.

### SUMMARY

When ultrasonic waves are oscillated from a vibrator to acquire an image with an ultrasound catheter, the ultrasonic waves are also oscillated from a side surface of the vibrator, in addition to a direction in which the vibrator faces. Therefore, some of the ultrasonic waves are directed in a direction different from a direction in which an image is desired to be captured, and are then reflected, leading to noise appearing on an ultrasonic image. Furthermore, this issue may cause a reduction in resolution of an ultrasonic image to be acquired. In particular, since an ultrasonic vibrator that generates ultrasonic waves at a low frequency is large in thickness, ultrasonic waves oscillated from a side surface increase, generating more noise.

The ultrasound catheter described here is capable of suppressing reflection of ultrasonic waves oscillated from a side surface of a vibrator, reducing noise in an ultrasonic image and improving resolution.
(1) An ultrasound catheter that achieves the object described above includes: an elongated sheath; an ultrasonic vibrator disposed inside the sheath; and a drive shaft that holds the ultrasonic vibrator inside the sheath and rotates the ultrasonic vibrator about a central axis of the sheath, in which the ultrasonic vibrator includes: an ultrasonic oscillation surface that is substantially parallel to the central axis of the sheath or inclined at an angle of 15 degrees or smaller with respect to the central axis of the sheath; a back surface that faces in a direction opposite to a direction in which the ultrasonic oscillation surface faces; and a side surface that is substantially orthogonal to the direction in which the ultrasonic oscillation surface faces, and, in the ultrasonic vibrator, an ultrasonic attenuation member covers the back surface and at least parts of lateral-direction side surface portions that the side surface includes and that mainly face in directions that are parallel to surface directions of the ultrasonic oscillation surface and that are orthogonal to directions in which the central axis of the sheath extends.

The ultrasound catheter configured as described above, in which, in the ultrasonic vibrator having a relatively greater thickness, the ultrasonic attenuation member covers the side surface mainly facing the directions that are parallel to the surface directions of the ultrasonic oscillation surface and that are orthogonal to the directions in which the central axis of the sheath extends, makes it possible to reduce noise in an ultrasonic image, which is to be generated due to ultrasonic waves oscillated from the side surface of the ultrasonic vibrator, making it possible to improve the ultrasonic image in resolution.
(2) In the ultrasound catheter according to (1) described above, in the ultrasonic vibrator, the ultrasonic attenuation member may cover at least a part of a rear side surface portion that the side surface includes and that mainly faces a proximal side of the sheath. As a result, it is possible to reduce noise in an ultrasonic image, which is to be generated due to ultrasonic waves oscillated from the rear side surface portion of the ultrasonic vibrator.
(3) In the ultrasound catheter according to (1) or (2) described above, in the ultrasonic vibrator, the ultrasonic attenuation member may cover at least a part of a front side surface portion that the side surface includes and that mainly faces a distal side of the sheath. As a result, it is possible to reduce noise in an ultrasonic image, which is to be generated due to ultrasonic waves oscillated from the front side surface portion of the ultrasonic vibrator.
(4) In the ultrasound catheter according to any one of (1) to (3) described above, in the ultrasonic vibrator, the surfaces other than the ultrasonic oscillation surface may be disposed and embedded in the ultrasonic attenuation member. As a result, since the ultrasonic attenuation member wholly covers the side surface of the ultrasonic vibrator, it is possible to further reduce an influence of the ultrasonic waves oscillated from the side surface of the ultrasonic vibrator on an ultrasonic image.
(5) In the ultrasound catheter according to any one of (1) to (4) described above, the ultrasonic vibrator may include a header portion on the rear side surface portion, the header portion being stepped down from the ultrasonic oscillation surface via a step portion that achieves reduction in thickness, and the ultrasonic attenuation member may include an attenuation member step portion that is continuous with the step portion and an attenuation member flat portion that is continuous with the header portion. As a result, it is possible to prevent the ultrasonic attenuation member from interfering with wires to the ultrasonic vibrator.
(6) In the ultrasound catheter according to any one of (1) to (5) described above, the ultrasonic vibrator may include a single element. As a result, it is possible to suppress generation of noise in an ultrasonic image, which may occur when ultrasonic waves oscillated from the ultrasonic vibrator interfere with each other or when ultrasonic waves oscillated from another ultrasonic vibrator are received.
(7) In the ultrasound catheter according to any one of (1) to (6) described above, in the ultrasonic vibrator, the ultrasonic attenuation member that is a single member may cover the back surface and at least a part of the side surface. As a result, the ultrasonic attenuation member is integrated, making it possible to make manufacturing of the ultrasonic attenuation member easier, more securely suppress noise in an ultrasonic image, and achieve improvement in resolution.
(8) In the ultrasound catheter according to any one of (1) to (7) described above, the ultrasonic vibrator may have a thickness falling within a range of 0.1 mm or thicker and 0.5 mm or thinner in directions orthogonal to the direction in which the ultrasonic oscillation surface faces. As a result, when an ultrasonic vibrator having a large thickness is used, it is possible to effectively attenuate ultrasonic waves to be oscillated from the side surface of the ultrasonic vibrator, making it possible to reduce an influence on an ultrasonic image.

According to another aspect, an ultrasound catheter comprises: an elongated sheath extending from a proximal end of the elongated sheath to a distal end of the elongated sheath; an axially movable and rotatable drive shaft positioned in the elongated sheath, the drive shaft having a distal end portion; and a vibration unit a positioned in the elongated sheath and fixed to the distal end portion of the drive shaft so that axial movement of the drive shaft results in axial movement of the vibration unit and rotation of the drive shaft results in rotation of the vibration unit. The vibration unit comprises an ultrasonic vibrator that oscillates ultrasonic waves and an ultrasonic attenuation member. The ultrasonic vibrator including: an ultrasonic oscillation surface from which the ultrasonic waves oscillates; a back surface facing in a direction opposite the ultrasonic oscillation surface; and a side surface extending between the ultrasonic oscillation surface and the back surface. The side surface of the ultrasonic vibrator including a front side surface portion that faces toward the distal end portion of the elongated sheath, and the side surface of the ultrasonic vibrator also including a rear side surface portion that faces away from the distal end portion of the elongated sheath. In addition, the ultrasonic vibrator is mounted on the ultrasonic attenuation member such that a part of the ultrasonic attenuation member covers the rear side surface portion of the side surface of the ultrasonic vibrator so that the ultrasonic attenuation member attenuates ultrasonic waves oscillated from the rear side surface portion of the side surface of the ultrasonic vibrator.

Another aspect involves an ultrasound catheter comprising: an elongated sheath extending from a proximal end of the elongated sheath to a distal end of the elongated sheath; an axially movable and rotatable drive shaft positioned in the elongated sheath, the drive shaft having a distal end portion; and a vibration unit a positioned in the elongated sheath and fixed to the distal end portion of the drive shaft so that axial movement of the drive shaft results in axial movement of the vibration unit and rotation of the drive shaft results in rotation of the vibration unit. The vibration unit includes an ultrasonic vibrator that oscillates ultrasonic waves and an ultrasonic attenuation member. The ultrasonic vibrator includes: an ultrasonic oscillation surface from which the ultrasonic waves oscillates; a back surface facing in a direction opposite the ultrasonic oscillation surface; and a side surface extending between the ultrasonic oscillation surface and the back surface, and defining an outer periphery of the ultrasonic vibrator. The side surface of the ultrasonic vibrator includes a front side surface portion that faces toward the distal end portion of the elongated sheath, with the side surface of the ultrasonic vibrator also including a rear side surface portion that faces away from the distal end portion of the elongated sheath. The ultrasonic attenuation member includes a recessed portion in which is positioned the ultrasonic vibrator so that: i) a bottom of the recessed portion faces the back surface of the ultrasonic vibrator; and ii) an upstanding side wall of the ultrasonic attenuation member projecting away from the bottom of the recessed portion faces towards and covers at least a part of the rear side surface portion of the side surface of the ultrasonic vibrator so that the ultrasonic attenuation member attenuates ultrasonic waves oscillated from the rear side surface portion of the side surface of the ultrasonic vibrator.

### BRIEF DESCRIPTION OF THE DRAWINGS

FIG. 1 is a front view illustrating an ultrasound catheter.

FIG. 2 is an enlarged cross-sectional view, when seen in a plan view, of a portion around a distal end portion of the ultrasound catheter.

FIG. 3 is an enlarged cross-sectional view, when seen in a front view, of a portion around the distal end portion of the ultrasound catheter.

FIG. 4 is an enlarged plan view of a vibrator unit.

FIG. 5 is a perspective view of an ultrasonic vibrator embedded in an ultrasonic attenuation member.

FIGS. 6A and 6B are images of stainless-steel pipes, which are acquired with the ultrasound catheter, in which FIG. 6A is an image in a case where no ultrasonic attenuation member is disposed to a side surface of an ultrasonic vibrator and FIG. 6B is an image in a case where an ultrasonic attenuation member is disposed to the side surface of the ultrasonic vibrator.

FIG. 7 is a schematic view illustrating a usage example of an ultrasound catheter system according to a present embodiment.

### DETAILED DESCRIPTION

Hereinafter, an embodiment of the ultrasound catheter disclosed here, representing one example of the new ultrasound catheter will be described with reference to the drawings. Dimensional ratios in the drawings may be exaggerated and different from actual ratios for convenience of description, illustration and understanding. Furthermore, in the present application, a side that is to be inserted into a living body is referred to as a “distal side” and a side on which an operation is to be performed is referred to as a “proximal side”.

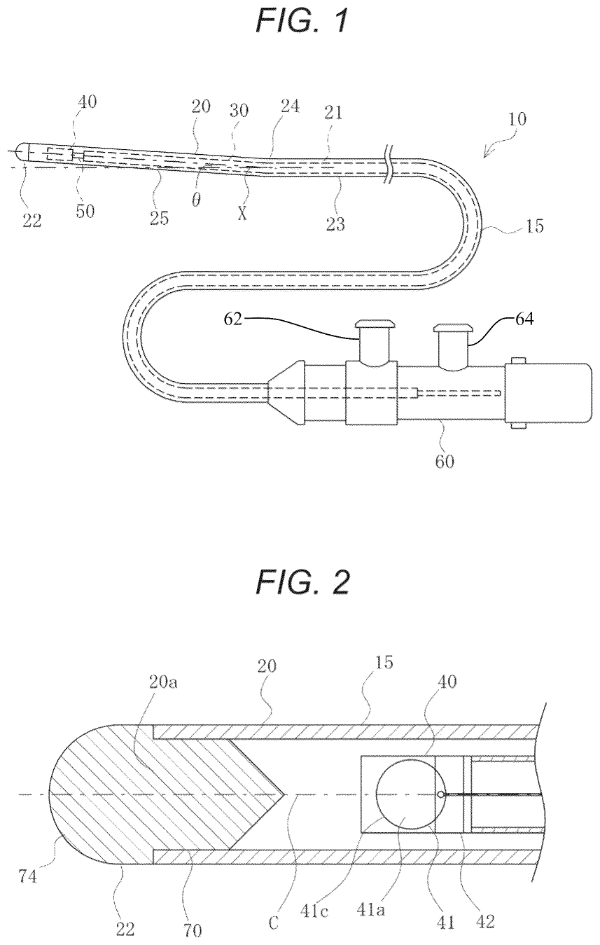

As illustrated in FIG. 1, an ultrasound catheter 10 according to the present embodiment includes a housing 60 at a proximal end portion of an elongated sheath 15. The sheath 15 includes an outer sheath 20 and an inner sheath 30, and a vibrator unit 40 and a drive shaft 50 that holds the vibrator unit 40 are disposed inside the sheath 15.

The outer sheath 20 is a tubular body to be inserted into a lumen in a living body. The outer sheath 20 includes a proximal tubular portion 23, a bent portion 24, and a distal tubular portion 25, when seen from proximal to distal. An accommodation lumen 21 communicating from a proximal end to a distal end is formed inside the proximal tubular portion 23, the bent portion 24, and the distal tubular portion 25.

The proximal tubular portion 23 is a tubular body having a substantially linear axial center. A proximal end portion of the proximal tubular portion 23 is fixed to the housing 60. The axial center of at least a distal end portion of the proximal tubular portion 23 is positioned on a linear reference line X. The bent portion 24 is a tubular body positioned distal of the proximal tubular portion 23 and having a bent axial center. The distal tubular portion 25 is a tubular body positioned distal of the bent portion 24 and having a linear axial center. A distal end portion of the distal tubular portion 25 is fixed to a cap 22.

A bending angle θ of the bent portion 24 is not limited in particular, but preferably ranges from 10° to 40° inclusive. If the bending angle θ is too small, an offset amount of a distal end portion of the outer sheath 20 with respect to a proximal end portion becomes small. The offset amount is a length from the reference line X to an axial center at a portion farthest from the reference line X in a direction perpendicular to the reference line X among portions on the outer sheath 20, which are positioned distal of the bent portion 24. If the bending angle θ is too large, rotation and movement in an axial center direction of the drive shaft 50 that rotates and moves in the axial center direction in a bent state inside the bent portion 24 are easily hindered. On the other hand, when the bending angle θ is an appropriate magnitude, it is easily possible to stably maintain rotation and movement in the axial center direction of the drive shaft 50 and to set the offset amount of the outer sheath 20 to a desired value.

A distal end portion length that is a length along the reference line X from a boundary between the proximal tubular portion 23 and the bent portion 24 to a most distal end of the outer sheath 20 is not limited in particular, but preferably ranges from 20 mm to 150 mm inclusive. Therefore, it is possible to appropriately set a length along the reference line X of a portion (portion where the axial center is shifted from the reference line X in a vertical direction of the reference line X) of the outer sheath 20, which is offset and distal of the bent portion 24, for use in a heart or a blood vessel having a wide lumen. Furthermore, if the distal end portion length is too short, the offset amount of the distal end portion of the outer sheath 20 with respect to the proximal end portion easily becomes small. If the distal end portion length is too long, the offset amount of the distal end portion of the outer sheath 20 with respect to the proximal end portion easily becomes large. On the other hand, when the distal end portion length is an appropriate length, it is easily possible to set the offset amount of the outer sheath 20 to a desired value.

The offset amount is not limited in particular, but preferably ranges from 5 mm to 30 mm inclusive. When the offset amount is an appropriate magnitude, it is easy to bring the outer sheath 20 close to an observation target portion in a heart or a blood vessel having a wide lumen.

The outer sheath 20 accommodates, in the accommodation lumen 21, the vibrator unit 40, the inner sheath 30, and the drive shaft 50. The vibrator unit 40, the inner sheath 30, and the drive shaft 50 in the outer sheath 20 are configured to move inside the accommodation lumen 21 along the axial center of the outer sheath 20. In addition, the vibrator unit 40 and the drive shaft 50 in the outer sheath 20 are configured to rotate about a central axis C of the outer sheath 15 inside the sheath 20. The outer sheath 20 is a cylindrical body that is only opened at its proximal end and is closed at its distal end by the cap 22. The proximal end portion of the proximal tubular portion 23, which forms the proximal end portion of the outer sheath 20, is fixed to the housing 60. A distal end portion of the distal tubular portion 25, which forms the distal end portion of the outer sheath 20, is fixed to the cap 22. The proximal end portion of the outer sheath 20 may be disposed with a reinforcement body such as a braided metal wire.

Although one bent portion 24 is disposed in the present embodiment, no bent portion may be disposed or two or more bent portions may be disposed.

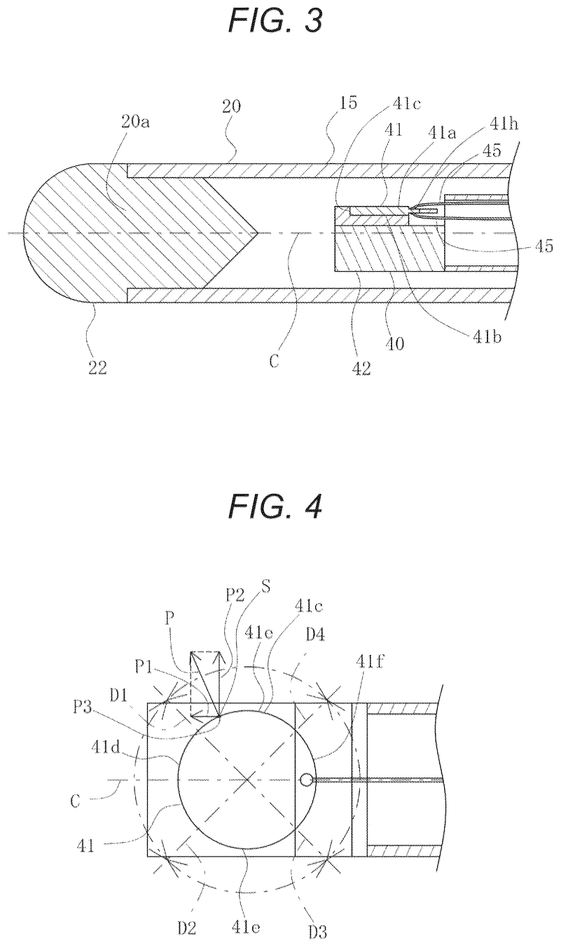

As illustrated in FIGS. 2 and 3, the vibrator unit 40 is disposed at a distal end portion of the sheath 15, and an opening portion 20a on a distal side of the outer sheath 20 forming the sheath 15 is sealed with the cap 22.

The vibrator unit 40 transmits and receives ultrasonic waves to and from biological tissue in the body. The vibrator unit 40 includes an ultrasonic vibrator 41 formed from a single element that transmits and receives ultrasonic waves and a vibrator holding portion 42 fixed to the drive shaft 50, in which the ultrasonic vibrator 41 is disposed. The vibrator unit 40 is configured to move inside the accommodation lumen 21 of the outer sheath 20 in the axial center direction of the outer sheath 20 beyond the bent portion 24. Furthermore, the vibrator unit 40 is configured to rotate about the axial center inside the accommodation lumen 21.

The ultrasonic vibrator 41 oscillates (produces) ultrasonic waves at a relatively low frequency ranging from 5 MHz to 20 MHz inclusive. A thickness of the ultrasonic vibrator 41 is relatively large, which ranges from 0.1 mm to 0.5 mm inclusive. Therefore, the ultrasonic vibrator 41 oscillates ultrasonic waves from not only an ultrasonic oscillation surface 41a but also a side surface 41c to some extent.

In the vibrator unit 40, the ultrasonic vibrator 41 includes the ultrasonic oscillation surface 41a that is substantially parallel to the central axis C of the sheath 15, a back surface 41b that faces in a direction opposite to a direction in which the ultrasonic oscillation surface 41a faces, and the side surface 41c that faces in directions orthogonal to the direction in which the ultrasonic oscillation surface 41a faces and directions in which the central axis C of the sheath 15 extends. As shown in FIG. 3, the side surface 41c of the ultrasonic vibrator 41 extends between and connects the back surface 41b and the ultrasonic oscillation surface 41a, and generally defines the outer perimeter of the ultrasonic vibrator 41. As illustrated in FIG. 4, the ultrasonic vibrator 41 has a circular shape, when seen in a plan view. A first line D1 and a second line D2 are assumed to each extend obliquely forward from a center position of the ultrasonic vibrator 41 to form an angle of 45°, and a third line D3 and a fourth line D4 are assumed to each extend obliquely backward from the center position of the ultrasonic vibrator 41 to form an angle of 45° with respect to the directions in which the central axis C of the sheath 15 extends. In this case, a region expanding from the first line D1 to the second line D2 in a counterclockwise direction in circumferential directions on the side surface 41c of the ultrasonic vibrator 41 is a front side surface portion 41d facing in a distal direction along the central axis C of the sheath 15. A region expanding from the third line D3 to the fourth line D4 in the counterclockwise direction in the circumferential directions on the side surface 41c of the ultrasonic vibrator 41 is a rear side surface portion 41f facing in a proximal direction along the central axis C of the sheath 15. A region expanding from the second line D2 to the third line D3 and a region expanding from the fourth line D4 to the first line D1 in the counterclockwise direction in the circumferential directions on the side surface 41c of the ultrasonic vibrator 41 are lateral-direction side surface portions 41e facing in directions orthogonal to the directions in which the central axis C of the sheath 15 extends.

On the side surface 41c of the ultrasonic vibrator 41, the lateral-direction side surface portions 41e mainly face in directions parallel to surface directions of the ultrasonic oscillation surface 41a and orthogonal to the directions in which the central axis C of the sheath 15 extends. The description here of mainly facing in the directions parallel to the surface directions of the ultrasonic oscillation surface 41a and orthogonal to the directions in which the central axis C of the sheath 15 extends means that, when a normal vector P is decomposed, at a desired point S on the side surface 41c of the ultrasonic vibrator 41, into a vector component P1 in one of the directions in which the central axis C of the sheath 15 extends, a vector component P2 in a direction orthogonal to the directions in which the central axis C of the sheath 15 extends and parallel to the surface directions of the ultrasonic oscillation surface 41a, and a vector component P3 in a direction orthogonal to the directions in which the central axis C of the sheath 15 extends and orthogonal to the vector component P2, the vector component P2 is largest.

On the side surface 41c of the ultrasonic vibrator 41, the front side surface portion 41d mainly faces a distal side of the sheath 15 and the rear side surface portion 41f mainly faces a proximal side of the sheath 15. The description here of mainly facing the distal side of the sheath 15 means that the vector component P1 faces the distal side and is largest among the vector components of the normal vector P described above. The description of mainly facing the proximal side of the sheath 15 means that the vector component P1 faces the proximal side and is largest among the vector components of the normal vector P described above. In the present embodiment, all tangential planes at the desired point S on the side surface 41c of the ultrasonic vibrator 41 are substantially orthogonal to the ultrasonic oscillation surface 41a. Therefore, as illustrated in FIG. 4, a magnitude of the vector component P3 of the normal vector P at the desired point S is always substantially zero. Furthermore, in the present embodiment, since the ultrasonic oscillation surface 41a is substantially parallel to the central axis C of the sheath 15, the vector component P3 is substantially orthogonal to the surface directions of the ultrasonic oscillation surface 41a.

The ultrasonic vibrator 41 may be inclined at an angle of 15° or smaller with respect to the central axis C of the sheath 15. The ultrasonic oscillation surface 41a is disposed at a position spaced apart outward in one of radial directions from the central axis C of the sheath 15.

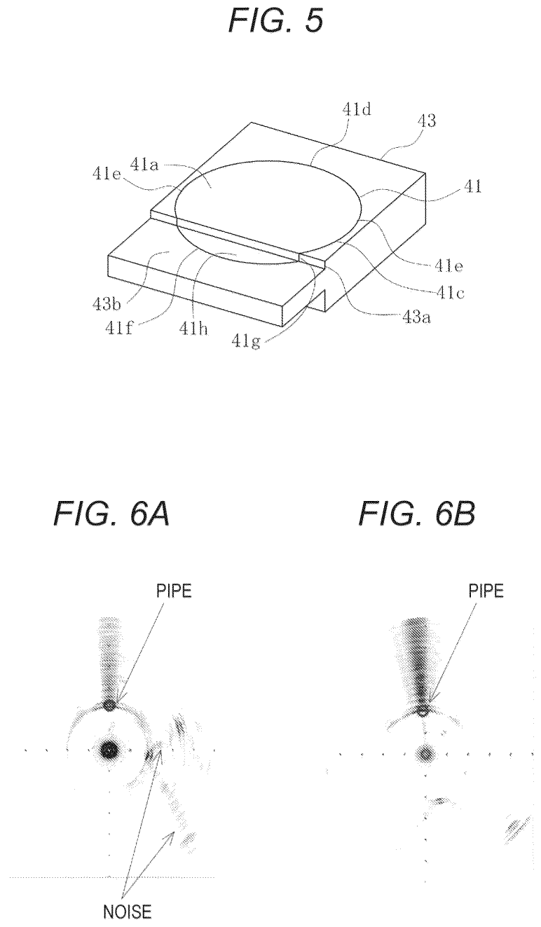

As illustrated in FIG. 5, the ultrasonic vibrator 41 is embedded in an ultrasonic attenuation member 43 including a single member (the ultrasonic attenuation member 43 is integrally formed as a single, one-piece part) to allow the back surface 41b that is opposite to the ultrasonic oscillation surface 41a and the side surface 41c to be wholly covered and to allow the ultrasonic oscillation surface 41a to be exposed. That is, in the ultrasonic vibrator 41, the surfaces other than the ultrasonic oscillation surface 41a, specifically, the back surface 41b and the side surface 41c are wholly disposed and embedded in the ultrasonic attenuation member 43. The ultrasonic attenuation member 43 offers a characteristic of attenuating ultrasonic waves that the ultrasonic vibrator 41 oscillates. The description referring to the “characteristic of attenuating ultrasonic waves” refers to a characteristic of allowing ultrasonic waves to be scattered or absorbed. As the ultrasonic attenuation member 43, it is possible to use, for example, but not limited to, an ultrasonic attenuation member in which a filler such as granular glass beads or polyethylene beads is mixed in a resin, and it is possible to use a desired material that attenuates ultrasonic waves.

The ultrasonic attenuation member 43 covers the front side surface portion 41d, the lateral-direction side surface portions 41e, and the rear side surface portion 41f of the side surface 41c of the ultrasonic vibrator 41. Stated differently, FIGS. 4 and 5 show that the ultrasonic vibrator 41 is positioned in a recessed portion of the ultrasonic attenuation member 43 such that the bottom of the recessed portion faces the back surface 41b of the ultrasonic vibrator 41, and a side wall of the ultrasonic attenuation member 43 extending upwardly away from bottom of the recessed portion faces towards and covers the side wall 41c of the ultrasonic vibrator 41. When a thickness of the ultrasonic attenuation member 43, at which the front side surface portion 41d and the lateral-direction side surface portions 41e are covered, is 0.25 mm or thicker at a thinnest portion, it is possible to sufficiently attenuate ultrasonic waves oscillated from the side surface 41c of the ultrasonic vibrator 41. The ultrasonic attenuation member 43 that covers the front side surface portion 41d of the ultrasonic vibrator 41 and that attenuates ultrasonic waves oscillated from the ultrasonic vibrator 41 toward the distal side makes it possible to reduce ring-shaped noise generated in an ultrasonic image. Furthermore, the ultrasonic attenuation member 43 that covers the lateral-direction side surface portions 41e of the ultrasonic vibrator 41 makes it possible to reduce noise appearing at a plurality of positions in the circumferential directions of an ultrasonic image when ultrasonic waves oscillated from the ultrasonic vibrator 41 in directions orthogonal to the central axis C are reflected toward the ultrasonic vibrator 41, making it possible to improve the ultrasonic image in resolution. In particular, since the ultrasonic vibrator 41 is configured to rotate about the central axis C of the sheath 15 and easily receives an influence of ultrasonic waves oscillated from the side surface 41c and ultrasonic waves that the side surface 41c receives, the ultrasonic attenuation member 43 makes it possible to reduce the influence.

The ultrasonic vibrator 41 includes, on the rear side surface portion 41f, a header portion 41h stepped down from the ultrasonic oscillation surface 41a via a step portion (step) 41g that achieves reduction in thickness. That is, the thickness of the header portion 41h is less than the thickness of the portion of the ultrasonic vibrator adjoining the header portion 41h (i.e., the thickness of the header portion 41h is less than the thickness of the remainder of the ultrasonic vibrator). The step portion 41g is formed on each of both the surfaces of the ultrasonic vibrator 41. Conductive wires 45 are coupled to both surfaces of the header portion 41h (see FIGS. 3 and 4). The ultrasonic attenuation member 43 includes an attenuation member step portion 43a that is continuous with the step portion 41g of the ultrasonic vibrator 41 and an attenuation member flat portion 43b that is continuous with the header portion 41h of the ultrasonic vibrator 41, and is thinned on its rear side in accordance with a shape of the ultrasonic vibrator 41. The ultrasonic attenuation member 43 having this shape is disposed to cover the rear side surface portion 41f of the ultrasonic vibrator 41 and to suppress interference with the conductive wires 45 coupled to the header portion 41h. The ultrasonic attenuation member 43 that covers the rear side surface portion 41f of the ultrasonic vibrator 41 makes it possible to reduce noise generated in an ultrasonic image when ultrasonic waves oscillated from the ultrasonic vibrator 41 toward the proximal side are reflected toward the ultrasonic vibrator 41.

The cap 22 is formed to include a radiopaque material and forms an imaging portion offering an X-ray contrast property. As the radiopaque material, it is possible to use, for example, barium sulfate, bismuth oxide, tungsten, gold, platinum, or tantalum. As illustrated in FIG. 2, the cap 22 includes an extension portion 70 extending from the opening portion 20a of the outer sheath 20 into the outer sheath 20 and a distal protruding portion 74 protruding from the opening portion 20a toward the distal side and having an outer diameter substantially identical to an outer diameter of the outer sheath 20.

The distal protruding portion 74 has a protruded dome shape protruding toward the distal side. Since the distal protruding portion 74 serves as a distal end of the ultrasound catheter 10, having the dome shape makes it possible to prevent biological tissue from being damaged.

As illustrated in FIG. 1, the inner sheath 30 is a cylindrical body including a part on the distal side, which is to be inserted into the outer sheath 20. A portion on the distal side of the inner sheath 30 is accommodated inside the outer sheath 20 and is configured to move along the axial center of the outer sheath 20. A proximal end portion of the inner sheath 30 is led out from (extends out from) the outer sheath 20 and the housing 60 toward the proximal side, and is fixed to a second housing (not illustrated).

The inner sheath 30 accommodates and allows the drive shaft 50 to rotate. A distal end portion of the inner sheath 30 is positioned close to the vibrator unit 40 on the proximal side of the vibrator unit 40. The inner sheath 30 is disposed between an inner peripheral surface of the outer sheath 20 and an outer peripheral surface of the drive shaft 50 to stabilize movement of the drive shaft 50 in rotational directions and axial directions.

The outer sheath 20 and the inner sheath 30 are not specifically limited in terms of the particular component material from which they may be made as long as the material is flexible and has strength to some extent, and it is possible to preferably use, for example, polyolefin such as polyethylene and polypropylene, polyamide, polyester such as polyethylene terephthalate, fluorine-based polymer such as polytetrafluoroethylene (PTFE) and ethylene tetrafluoroethylene copolymer (ETFE), polyetheretherketone (PEEK), and polyimide.

The drive shaft 50 transmits, to the vibrator unit 40, a rotational force and a moving force in the axial center direction, which act from a drive unit (not illustrated). When the drive shaft 50 transmits power of rotation, the vibrator unit 40 rotates, making it possible to observe an internal structure of tissue from a blood vessel or a heart chamber in a 360-degree manner. Furthermore, the drive shaft 50 is configured to move inside the accommodation lumen 21 of the outer sheath 20 along the axial center (axial direction movement) of the outer sheath 20.

The housing 60, to which the proximal end portion of the outer sheath 20 is liquid-tightly secured, includes a first port 62 and a second port 64. The housing 60 is not limited in terms of the particular component material from which it may be fabricated, as long as the material has strength to some extent, and it is possible to preferably use, for example, polycarbonate, polyamide, polysulfone, polyarylate, or a methacrylate-butylene-styrene copolymer.

Ultrasonic scanning with the ultrasound catheter 10 is performed as rotational movement is transmitted to the drive shaft 50 and the vibrator unit 40, which is fixed to a distal end of the drive shaft 50, is also rotated. As a result, it is possible to perform scanning with ultrasonic waves transmitted and received to and from the ultrasonic vibrator 41 substantially in the radial directions. In addition, pulling the drive shaft 50 toward the proximal side makes it possible to rotate and move the ultrasonic vibrator 41 toward the proximal side. Therefore, it is possible to acquire in a scanning manner a 360° cross-sectional image of surrounding tissue of a blood vessel or a heart chamber to a desired position along the axial center of the outer sheath 20.

To demonstrate an example of advantageous results associated with the ultrasonic catheter disclosed here, a stainless-steel pipe having a diameter of 20 mm was disposed near the distal end portion of the sheath 15, and imaging was performed in respective cases when the side surface 41c of the ultrasonic vibrator 41 was covered and was not covered with the ultrasonic attenuation member 43. As illustrated in FIG. 6A, when the side surface 41c of the ultrasonic vibrator 41 was not covered with the ultrasonic attenuation member 43, noise occurred at a plurality of positions in the circumferential directions in an ultrasonic image. As illustrated in FIG. 6B, when the side surface 41c of the ultrasonic vibrator 41 was covered with the ultrasonic attenuation member 43, noise was reduced in the ultrasonic image. Furthermore, it was also confirmed that the ultrasonic image was higher in resolution when the side surface 41c of the ultrasonic vibrator 41 was covered with the ultrasonic attenuation member 43, than the ultrasonic image when the side surface was not covered with the ultrasonic attenuation member.

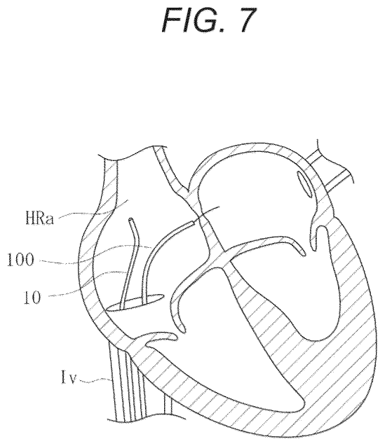

As illustrated in FIG. 7, for example, it is possible to insert the ultrasound catheter 10 according to the present embodiment from a femoral vein Iv into a right atrium HRa for use in observing a treatment situation using a therapeutic catheter 100.
(1) As described above, the ultrasound catheter 10 according to the present embodiment includes: the elongated sheath 15; the ultrasonic vibrator 41 disposed inside the sheath 15; and the drive shaft 50 that holds the ultrasonic vibrator 41 inside the sheath 15 and rotates the ultrasonic vibrator 41 about the central axis C of the sheath 15, in which the ultrasonic vibrator 41 includes: the ultrasonic oscillation surface 41a that is substantially parallel to the central axis C of the sheath 15 or inclined at an angle of 15 degrees or smaller with respect to the central axis C of the sheath 15; the back surface 41b that faces in a direction opposite to a direction in which the ultrasonic oscillation surface 41a faces; and the side surface 41c that is substantially orthogonal to the direction in which the ultrasonic oscillation surface 41a faces, and, in the ultrasonic vibrator 41, the ultrasonic attenuation member 43 covers the back surface 41b and at least parts of the lateral-direction side surface portions 41e that the side surface 41c includes and that mainly face in directions that are parallel to surface directions of the ultrasonic oscillation surface 41a and that are orthogonal to directions in which the central axis C of the sheath 15 extends. The ultrasound catheter configured as described above, in which, in the ultrasonic vibrator 41 having a relatively greater thickness, the ultrasonic attenuation member 43 covers the side surface 41c mainly facing the directions that are parallel to the surface directions of the ultrasonic oscillation surface 41a and that are orthogonal to the directions in which the central axis C of the sheath 15 extends, makes it possible to reduce noise to be generated in an ultrasonic image due to ultrasonic waves oscillated from the side surface 41c of the ultrasonic vibrator 41, making it possible to improve the ultrasonic image in resolution.
(2) In the ultrasound catheter 10 according to (1) described above, in the ultrasonic vibrator 41, the ultrasonic attenuation member 43 may cover at least a part of the rear side surface portion 41f that the side surface 41c includes and that mainly faces a proximal side of the sheath 15. As a result, it is possible to reduce noise in an ultrasonic image, which is to be generated due to ultrasonic waves oscillated from the rear side surface portion 41f of the ultrasonic vibrator 41.
(3) In the ultrasound catheter 10 according to (1) or (2) described above, in the ultrasonic vibrator 41, the ultrasonic attenuation member 43 may cover at least a part of the front side surface portion 41d that the side surface 41c includes and that mainly faces a distal side of the sheath 15. As a result, it is possible to reduce noise in an ultrasonic image, which is to be generated due to ultrasonic waves oscillated from the front side surface portion 41d of the ultrasonic vibrator 41.
(4) In the ultrasound catheter 10 according to any one of (1) to (3) described above, in the ultrasonic vibrator 41, the surfaces other than the ultrasonic oscillation surface 41a may be disposed and embedded in the ultrasonic attenuation member 43. As a result, since the ultrasonic attenuation member 43 wholly covers the side surface 41c of the ultrasonic vibrator 41, it is possible to further reduce an influence of the ultrasonic waves oscillated from the side surface 41c of the ultrasonic vibrator 41 on an ultrasonic image.
(5) In the ultrasound catheter 10 according to any one of (1) to (4) described above, the ultrasonic vibrator 41 may include the header portion 41h on the rear side surface portion 41f, the header portion 41h being stepped down from the ultrasonic oscillation surface 41a via the step portion 41g that achieves reduction in thickness, and the ultrasonic attenuation member 43 may include the attenuation member step portion 43a that is continuous with the step portion 41g and the attenuation member flat portion 43b that is continuous with the header portion 41h. As a result, it is possible to prevent the ultrasonic attenuation member 43 from interfering with wires to the ultrasonic vibrator 41.
(6) In the ultrasound catheter 10 according to any one of (1) to (5) described above, the ultrasonic vibrator 41 may include a single element. As a result, it is possible to suppress generation of noise in an ultrasonic image, which may occur when ultrasonic waves oscillated from the ultrasonic vibrator 41 interfere with each other or when ultrasonic waves oscillated from another ultrasonic vibrator 41 are received.
(7) In the ultrasound catheter 10 according to any one of (1) to (6) described above, in the ultrasonic vibrator 41, the ultrasonic attenuation member 43 that is a single member may cover the back surface 41b and at least a part of the side surface 41c. As a result, the ultrasonic attenuation member 43 is integrated, making it possible to make manufacturing of the ultrasonic attenuation member 43 easier, more securely suppress noise in an ultrasonic image, and achieve improvement in resolution.
(8) In the ultrasound catheter 10 according to any one of (1) to (7) described above, the ultrasonic vibrator 41 may have a thickness falling within a range of 0.1 mm or thicker and 0.5 mm or thinner in directions orthogonal to the direction in which the ultrasonic oscillation surface 41a faces. As a result, when an ultrasonic vibrator 41 having a large thickness is used, it is possible to effectively attenuate ultrasonic waves to be oscillated from the side surface 41c of the ultrasonic vibrator 41, making it possible to reduce an influence on an ultrasonic image.

The detailed description above describes embodiments of an ultrasonic catheter representing an example of the new ultrasonic catheter disclosed here. The invention is not limited, however, to the precise embodiments and variations described. Various changes, modifications and equivalents can be effected by one skilled in the art without departing from the spirit and scope of the invention as defined in the accompanying claims. It is expressly intended that all such changes, modifications and equivalents that fall within the scope of the claims are embraced by the claims.

What is claimed is:

1. An ultrasound catheter comprising:
an elongated sheath;
an ultrasonic vibrator disposed inside the sheath;
a drive shaft that holds the ultrasonic vibrator inside the sheath and rotates the ultrasonic vibrator about a central axis of the sheath;
the ultrasonic vibrator including: an ultrasonic oscillation surface that is substantially parallel to the central axis of the sheath or inclined at an angle of 15 degrees or smaller with respect to the central axis of the sheath; a back surface that faces in a direction opposite to a direction in which the ultrasonic oscillation surface faces; and a side surface that is substantially orthogonal to the direction in which the ultrasonic oscillation surface faces; and
in the ultrasonic vibrator, an ultrasonic attenuation member covers the back surface and at least parts of lateral-direction side surface portions that the side surface includes and that mainly face in directions that are parallel to surface directions of the ultrasonic oscillation surface and that are orthogonal to directions in which the central axis of the sheath extends.

2. The ultrasound catheter according to claim 1, wherein the side surface of the ultrasonic vibrator includes a rear side surface portion that mainly faces a proximal side of the sheath, the side surface of the ultrasonic vibrator also including a front side surface portion facing in a direction opposite the rear side surface portion, the ultrasonic attenuation member covering at least a part of the rear side surface portion of the ultrasonic vibrator.

3. The ultrasound catheter according to claim 2, wherein the ultrasonic attenuation member covers at least a part of the front side surface portion of the ultrasonic vibrator that mainly faces a distal side of the sheath.

4. The ultrasound catheter according to claim 3, wherein the rear side surface of the ultrasonic vibrator and at least a part of the side surface of the ultrasonic vibrator are embedded in the ultrasonic attenuation member.

5. The ultrasound catheter according to claim 4, wherein
the ultrasonic vibrator includes a header portion on the rear side surface portion, the header portion being stepped down from the ultrasonic oscillation surface via a step portion so that a thickness of the header portion is less than the thickness of a portion of the ultrasonic vibrator adjoining the header portion, and
the ultrasonic attenuation member includes an attenuation member step portion that is continuous with the step portion of the ultrasonic vibrator and an attenuation member flat portion that is continuous with the header portion.

6. The ultrasound catheter according to claim 5, wherein the ultrasonic vibrator is a single element.

7. The ultrasound catheter according to claim 5, wherein the ultrasonic attenuation member is a single member and covers the back surface of the ultrasonic vibrator and at least a part of the side surface of the ultrasonic vibrator.

8. The ultrasound catheter according to claim 5, wherein the ultrasonic vibrator has a thickness within a range of 0.1 mm or thicker and 0.5 mm or thinner in directions orthogonal to the direction in which the ultrasonic oscillation surface faces.

9. The ultrasound catheter according to claim 1, wherein the ultrasonic vibrator is a single element.

10. The ultrasound catheter according to claim 1, wherein the ultrasonic attenuation member is a single member and covers the back surface of the ultrasonic vibrator and at least a part of the side surface of the ultrasonic vibrator.

11. The ultrasound catheter according to claim 1, wherein the ultrasonic vibrator has a thickness within a range of 0.1 mm or thicker and 0.5 mm or thinner in directions orthogonal to the direction in which the ultrasonic oscillation surface faces.

12. An ultrasound catheter comprising:
an elongated sheath extending from a proximal end of the elongated sheath to a distal end of the elongated sheath;
an axially movable and rotatable drive shaft positioned in the elongated sheath, the drive shaft having a distal end portion;
a vibration unit a positioned in the elongated sheath and fixed to the distal end portion of the drive shaft so that axial movement of the drive shaft results in axial movement of the vibration unit and rotation of the drive shaft results in rotation of the vibration unit;
the vibration unit comprising an ultrasonic vibrator that oscillates ultrasonic waves and an ultrasonic attenuation member;
the ultrasonic vibrator including: an ultrasonic oscillation surface from which the ultrasonic waves oscillates; a back surface facing in a direction opposite the ultrasonic oscillation surface; and a side surface extending between the ultrasonic oscillation surface and the back surface;
the side surface of the ultrasonic vibrator including a front side surface portion that faces toward the distal end portion of the elongated sheath, the side surface of the ultrasonic vibrator also including a rear side surface portion that faces away from the distal end portion of the elongated sheath; and
the ultrasonic vibrator being mounted on the ultrasonic attenuation member such that a part of the ultrasonic attenuation member covers the rear side surface portion of the side surface of the ultrasonic vibrator so that the ultrasonic attenuation member attenuates ultrasonic waves oscillated from the rear side surface portion of the side surface of the ultrasonic vibrator.

13. The ultrasound catheter according to claim 12, wherein the ultrasonic oscillation surface is parallel to the central axis of the sheath or is inclined at an angle of 15 degrees or less with respect to a central axis of the sheath.

14. The ultrasound catheter according to claim 12, wherein the ultrasonic vibrator is mounted on the ultrasonic attenuation member such that a part of the ultrasonic attenuation member covers the front side surface portion of the side surface of the ultrasonic vibrator so that the ultrasonic attenuation member attenuates ultrasonic waves oscillated from the front side surface portion of the side surface of the ultrasonic vibrator.

15. The ultrasound catheter according to claim 12, wherein the distal end portion of the elongated sheath is an open end, and further comprising a cap fixed to the distal end portion of the elongated sheath to close the open end of the elongated sheath.

16. The ultrasound catheter according to claim 12, wherein the rear side surface portion of the ultrasonic vibrator is a header portion having a thickness less than the thickness of a portion of the ultrasonic vibrator adjoining the header portion, and the ultrasonic attenuation member includes an attenuation member flat portion surrounding the header portion and having a thickness less that the thickness of a portion of the ultrasonic attenuation member adjoining the attenuation member flat portion.

17. The ultrasound catheter according to claim 16, further comprising conductive wires coupled to the header portion of the ultrasonic vibrator having the thickness less than the thickness of the portion of the ultrasonic vibrator adjoining the header portion.

18. An ultrasound catheter comprising:
an elongated sheath extending from a proximal end of the elongated sheath to a distal end of the elongated sheath;
an axially movable and rotatable drive shaft positioned in the elongated sheath, the drive shaft having a distal end portion;
a vibration unit a positioned in the elongated sheath and fixed to the distal end portion of the drive shaft so that axial movement of the drive shaft results in axial movement of the vibration unit and rotation of the drive shaft results in rotation of the vibration unit;
the vibration unit comprising an ultrasonic vibrator that oscillates ultrasonic waves and an ultrasonic attenuation member;
the ultrasonic vibrator including: an ultrasonic oscillation surface from which the ultrasonic waves oscillates; a back surface facing in a direction opposite the ultrasonic oscillation surface; and a side surface extending between the ultrasonic oscillation surface and the back surface, and defining an outer periphery of the ultrasonic vibrator;
the side surface of the ultrasonic vibrator including a front side surface portion that faces toward the distal end portion of the elongated sheath, the side surface of the ultrasonic vibrator also including a rear side surface portion that faces away from the distal end portion of the elongated sheath; and
the ultrasonic attenuation member including a recessed portion in which is positioned the ultrasonic vibrator so that: i) a bottom of the recessed portion faces the back surface of the ultrasonic vibrator; and ii) an upstanding side wall of the ultrasonic attenuation member projecting away from the bottom of the recessed portion faces towards and covers at least a part of the rear side surface portion of the side surface of the ultrasonic vibrator so that the ultrasonic attenuation member attenuates ultrasonic waves oscillated from the rear side surface portion of the side surface of the ultrasonic vibrator.

19. The ultrasound catheter according to claim 18, wherein the ultrasonic oscillation surface is parallel to the central axis of the sheath or is inclined at an angle of 15 degrees or less with respect to a central axis of the sheath.

20. The ultrasound catheter according to claim 18, wherein the upstanding side wall of the ultrasonic attenuation member also faces towards and covers at least a part of the front side surface portion of the side surface of the ultrasonic vibrator.
